(19) United States
(12) Patent Application Publication
(10) Pub. No.: US 2024/0173315 A1
(43) Pub. Date: May 30, 2024

(54) TRAZODONE ORAL SOLUTION

(71) Applicant: OWP Pharmaceuticals, Inc., Naperville, IL (US)

(72) Inventors: Paul Sudhakar, Shawnee, KS (US); Scott Boyer, West Chicago, IL (US)

(21) Appl. No.: 18/508,992

(22) Filed: Nov. 14, 2023

Related U.S. Application Data
(60) Provisional application No. 63/383,547, filed on Nov. 14, 2022.

(51) Int. Cl. A61K 31/496; A61K 9/00; A61K 9/08; A61K 47/12; A61K 47/14; A61K 47/18; A61K 47/22; A61K 47/26; A61K 47/46

(52) U.S. Cl. CPC A61K 31/496; A61K 9/0053; A61K 9/08; A61K 47/12; A61K 47/14; A61K 47/183; A61K 47/22; A61K 47/26; A61K 47/46

(57) ABSTRACT

The present invention provides for an oral solution containing trazodone, methods of manufacturing the same, and methods of using the same, e.g., to treat major depressive disorder (MDD).

This application claims the benefit of priority to U.S. Provisional Patent Application No. 63/383.547, filed Nov. 14, 2022, which is hereby incorporated by reference herein.

### BACKGROUND

Trazodone, or 2-[3-[4-(3-chlorophenyl)-1-piperazinylpropyl]-1,2,4-triazolo[4,3-a]pyridin-3(2H)-one, is an antidepressant which, though having a significant effect on the serotonin receptors, is neither a psychostimulant, nor a MAO inhibitor, nor a tricyclic antidepressant. Furthermore, trazodone possesses analgesic properties.

Trazodone alleviates the characteristic symptoms of depression, in particular anxiety, somatization, psychomotor retardation, hypochondria, mood swings, irritability, insomnia, apathy, feeling of fatigue and lack of energy, depressed mood.

Trazodone has also proved effective in controlling pronounced essential tremor, probably on account of its serotoninergic activity.

Moreover, the antidepressant and anxiolytic properties of trazodone have proved useful in the treatment of symptoms of withdrawal from cocaine, benzodiazepines, and alcohol. Besides the above-mentioned activities, its sleep-inducing activity is also known.

Trazodone is typically used medically in the form of a pharmaceutically acceptable salt of acid addition. The preferred form is the hydrochloride salt obtained by treatment of the free base with hydrochloric acid.

Desyrel® (trazodone hydrochloride) is available as scored oral tablets in 50 mg, 100 mg, 150 mg, and 300 mg and is indicated for the treatment of major depressive disorder (NIDD).

Although oral solid dosage forms such as tablets are very popular for reasons that are mainly due to ease of management, for certain users (e.g., children and the elderly) these forms are not necessarily a convenient option, especially due to difficulty in &wallowing these forms. This lack of convenience results in a high incidence of non-compliance and ineffective therapy. It is also difficult to appropriately titrate, even as a scored tablet.

### SUMMARY

The present invention provides for an oral solution that includes: (i) trazodone; buffer, (iii) sweetener, (iv) preservative, (v) chelating agent, (vi) colorant, (vii) flavor, and (viii) solvent.

The present invention also provides for an oral solution that includes: (i) 16.44±1.6 mg/ml trazodone; (ii) 2.40±0.2 mg/ml buffer, (iii) 251.5±25 mg/ml sweetener, (iv) 2±0.2 mg/ml preservative, (v) 3±0.3 mg/ml chelating agent, (vi) 0.01±0.001 mg/ml colorant, (vii) 2±0.2 mg/ml flavor, and (viii) 739.09±73 mg/ml solvent.

The present invention also provides for an oral solution that includes: (i) 16.44 mg/ml trazodone; (ii) 2.40 mg/ml buffer, (iii) 251.5 mg/ml sweetener, (iv) 2 mg/ml preservative, (v) 3 mg/ml chelating agent, (vi) 0.01 mg/ml colorant, (vii) 2 mg/ml flavor, and (viii) 739.09 mg/ml solvent.

The present invention also provides for an oral solution that includes: (i) trazodone; (ii) citric acid, anhydrous; (iii) sorbitol solution, 70% (w/v); (iv) sodium saccharin; (v) sodium benzoate, powder; (vi) methylparaben, powder; (vii) edetate disodiurn dihydrate; (viii) FD&C Red No. 40; (ix) cherry flavor; and (x) purified water.

The present invention also provides for an oral solution that includes: (i) 16.44±1.6 mg/ml trazodone; (ii) 2.40±0.2 mg/ml citric acid, anhydrous; (iii) 250±25 mg/ml sorbitol solution, 70% (w/v); (iv) 1.50±0.15 mg/ml sodium saccharin; (v) 1±0.1 mg/ml sodium benzoate, powder; (vi) 1±0.1 mg/ml methylparaben, powder; (vii) 3±0.3 mg/ml edetate di sodium dihydrate; (viii) 0.01±0.001 mg/ml FD&C Red No. 40; (ix) 2±0.2 mg/ml cherry favor; and (x) 739.09±75 mg/ml purified water.

The present invention also provides for an oral solution that includes: (i) 16.44 mg/ml trazodone; (ii) 2.40 mg/ml citric acid, anhydrous; (iii) 250 mg/ml sorbitol solution, 70% (w/v); (iv) 1.50 mg/ml sodium saccharin; (v) 1 mg/ml sodium benzoate, powder; (vi) 1 mg/ml methylparaben, powder; (vii) 3 mg/ml edetate disodium dihydrate; (viii) 0.01 mg/ml FD&C Red No. 40; (ix) 2 mg/ml cherry flavor; and (x) 739.09 mg/ml purified water.

The present invention also provides for a method of treating in a human subject at least one of major depressive disorder, anxiety disorder, and difficulty with sleep. The method includes administering to the human subject in need thereof an affective amount of the oral solution described herein.

### BRIEF DESCRIPTION OF THE FIGURE

FIG. 1 illustrates manufacturing process and controls for a trazodone oral solution,

### DETAILED DESCRIPTION

The present invention can be more readily understood by reading the following detailed description of the invention and study of the included examples.

As used herein, the following terms have the meaning ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The articles “a” and “an” as used herein refer to “one or more” or “at least one,” unless otherwise indicated. That is, reference to any element or component of an embodiment by the indefinite article “a” “an” does not exclude the possibility that more than one element or component is present.

As used herein, “trazodone” refers to the compound having the chemical name 2-{3-[4-(3-chlorophenyl)piperazin-1-yl]propyl}[1,2,4]triazolo[4,3-a]pyridin-3(2H)-one; molecular formula C₁₉H₂₂ClN₅O; CAS number 19794-93-5; and molar mass 371.87 g·mol⁻¹. Trazodone is an antidepressant, used to treat, e.g., major depressive disorder, anxiety disorders, and difficulties with sleep. The chemical structure is shown below.

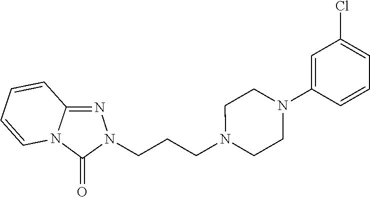 [SMILES: O=c1n(CCCN2CCN(c3cccc(Cl)c3)CC2)nc2ccccn12]

Trazodone can also exist as a pharmaceutically acceptable salt, e.g., trazodone hydrochloride. Trazodone hydrochloride has the FDA unique ingredient identifier (UNII) 6E8ZO8LRNM. Unless expressly stated otherwise, reference to trazodone is a reference to the free base, as well as a pharmaceutically acceptable salt thereof (e.g., trazodone hydrochloride).

The term “pharmaceutically acceptable” refers to those compounds, excipients, active ingredients, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

As used herein, “oral solution” refers to the oral liquid dosage foim in which the active ingredient is mixed with a liquid, while being dissolved and/or dispersed therein. The active ingredient particles are substantially dissolved and/or dispersed in the liquid. Typically, oral solutions are intended for enteral delivery by orally administering, with or without food.

As used herein, “buffer” or “buffering agent” refers to a substance capable of keeping the pH at a nearly constant value in a wide variety of chemical applications. Typically, the buffer consists of a mixture of a weak acid and its conjugate base, or vice versa. One buffer suitable in the manufacture of the oral solution described herein is citric acid (e.g., citric acid anhydrous).

As used herein, “sweetener” or “sweetening agent” refers to a substance that when added to the oral solution, imparts the flavor of sweetness, either because it contains a type of sugar, or because it contains a sweet-tasting sugar substitute. Sweeteners suitable in the manufacture of the oral solution described herein include sorbitol (e.g., sorbitol solution, 70%) and saccharin (e.g., sodium saccharin).

As used herein, “preservative” or “antimicrobial preservative” refers to a substance that when added to the oral solution, prevents, or mitigates decomposition by microbial growth or by undesirable chemical changes. Preservatives suitable in the manufacture of the oral solution described herein include sodium benzoate (e.g., sodium benzoate, powder) and methyl paraben (e.g., methylparaben, powder).

As used herein, “chelating agent” refers to a substance capable of forming a water-soluble complex. One chelating agent suitable in the manufacture of the oral solution described herein is EDTA (e.g., edetate disodium dihydrate).

As used herein, “colorant” or “coloring agent” refers to a substance that is added in order to change the color of the oral solution. One colorant suitable in the manufacture of the oral solution described herein is FD&C Red No. 40.

As used herein, “flavor” or “flavoring agent” refers to a substance that is added to improve the taste or smell of the oral solution. One flavor suitable in the manufacture of the oral solution described herein is cherry flavor (e.g., cherry flavor, natural & artificial).

As used herein, “solvent” refers to a liquid substance capable of effectively dissolving and/or dispersing the active ingredient trazodone, or pharmaceutically acceptable salt thereof, and the excipients present in the oral solution described herein. One solvent suitable in the manufacture of the oral solution described herein is water (e.g., purified water).

As used herein, “citric acid anhydrous” refers to citric acid that is substantially water-free. Citric acid refers to the compound with the chemical name 2-hydroxypropane-1,2,3-tricarboxylic acid; CAS number 77-92-9; chemical formula C611807; and molar mass 192.123 g·mol⁻¹, Citric acid anhydrous has the FDA unique ingredient identifier (UNII) XF417D3PSL.

As used herein, “sorbitol solution 70 percent” refers to a liquid solution containing 70% (w/v) sorbitol. Sorbitol refers to the compound with the chemical name (2S,3R,4R,5R)-hexane-1,2,3,45,6-hexol, CAS number 50-70-4; chemical formula C₆H₁₄O₆; and molar mass 182.17 gm·mol⁻¹. Sorbitol solution, 70 percent has the FDA unique ingredient identifier (UNII) 8KW3E207O2.

As used herein, “sodium saccharin” refers to the sodium salt of saccharin, which is the compound having the chemical name 1H-1λ^{6,}2-benzothiazole-1,1,3(2H)-trione. Saccharin has the CAS number 81-07-2; chemical formula C₇H₅NO₃S; and molar mass 183.18 g·mol⁻¹. Sodium saccharin has the FDA unique ingredient identifier (UNII) SB8ZUX40TY.

As used herein. “sodium benzoate powder” rders to sodium benzoate in a powder form. Sodium benzoate has the CAS number 532-32-1; chemical formula C₇H₅NO₃S; and molar mass 144.105 g·mol⁻¹. Sodium benzoate, powder has the FDA unique ingredient identifier (UNII) OJ245FE5EU.

As used herein, “methylparaben powder” refers to methylparaben in a powder form. Methylparaben has the chemical name methyl 4-hydroxybenzoate; CAS number 99-76-3; chemical formula C₈H₈O₃; and molar mass 152.149 g·mol⁻¹. Methylparaben, powder has the FDA unique ingredient identifier (UNII) A218C7HI9T.

As used herein, “ethylenediaminetetraacetic acid” or “EDTA” refers to the compound having the chemical name 2,2′,2″,2′″-(ethane-1,2-diyldinitrilo)tetraacetic acid; CAS number 60-00-4 (free acid), CAS number 6381-92-6 (dihydrate disodium salt); chemical formula C₁₀H₁₆N₂O₈; and molar mass 292.244 g·mol⁻¹. Edetate disodium dihydrate has the FDA unique ingredient identifier (UNII) 7FED91C86K.

The terin “treating” (and equivalent terms such as “treat,” “treated,” and “treatment”) of a subject includes the administration of an active pharmaceutical ingredient (API), or a unit dosage form containing the same (e.g., oral solution), to a subject with the purpose of preventing, mitigating, curing, healing, alleviating, relieving, altering, remedying, ameliorating, improving, stabilizing or affecting a disease or disorder, or a symptom of the disease or disorder. The disease or disorder can be, e.g., major depressive disorder (MDD).

As used herein, “major depressive disorder” or “MDD” (also known as clinical depression) refers to a mental disorder characterized by at least two weeks of pervasive low mood, low self-esteem, and loss of interest or pleasure in normally enjoyable activities.

As used herein, “anxiety disorder” refers to a cluster of mental disorders characterized by significant and uncontrollable feelings of anxiety and fear such that a person's social, occupational, and personal function are significantly impaired. Anxiety may cause physical and cognitive symptoms, such as restlessness, irritability, easy fatiguability, difficulty concentrating, increased heart rate, chest pain, abdominal pain, and a variety of other symptoms that may vary based on the individual.

As used herein, “difficulty with sleep” or “sleep disorder” (also known as somnipathy) refers to a medical disorder of an individual's sleep patterns. Some sleep disorders are severe enough to interfere with normal physical, mental, social, and emotional functioning. Sleep disorders are broadly classified into dyssomnias, parasomnias, circadian rhythm sleep disorders involving the timing of sleep, and other disorders including ones caused by medical or psychological conditions. When a person struggles to fall asleep and/or stay asleep with no obvious cause, it is referred to as insomnia, the most common sleep disorder. Others include sleep apnea, narcolepsy, and hypersomnia (excessive sleepiness at inappropriate times), sleeping sickness (disruption of sleep cycle due to infection), sleepwalking, and night terrors.

The term “oral administration” or “PO” refers to a route of administration where a substance is taken through the mouth. Many medications are taken orally because they are intended to have a systemic effect, reaching different parts of the body via the bloodstream.

The oral solution dosage form provided for herein may be prepared, e.g., by contacting the ingredient(s) with the solvent. In doing so, any one or more of the ingredients employed (active ingredient and/or excipients) can effectively be dissolved, suspended, or dispersed therein (e.g, in the solvent). This includes, e.g., salts, solvates, hydrates, dihydrates, monohydrates, powders, microcrystalline forms, crystalline forms, amorphous forms, specified particle size distribution (ND), etc. of the ingredients. In doing so, the ingredient would therefore no longer necessarily retain that specified form. However, within the context of the present invention, it is appreciated that those of skill in the art understand and agree that reference to the resulting oral solution dosage form as containing ingredients in the indicated form is otherwise acceptable and appropriate.

Additionally, within the context of the present invention, it is appreciated that those of skill in the art understand and agree that reference to the oral solution dosage form as containing the ingredients having a specified state is acceptable and appropriate. This is so, even though those ingredients may no longer necessarily exist in the same state (as specifically indicated) as when introduced to the solvent. Likewise, within the context of the present invention, reference can also be made to the oral solution dosage form as being manufactured from (or as being formed from) the ingredients, as specifically indicated. It is appreciated that those of skill in the art understand and agree that each of the above characterizations of the oral solution dosage form are acceptable and appropriate.

For example, within the context of the oral solution dosage form, reference to “citric acid anhydrous” is also a reference to citric acid. Within the context of the oral solution dosage form, reference to “sorbitol solution, 70 percent” is also a reference to sorbitol. Within the context of the oral solution dosage form, reference to “sodium saccharin” or “saccharin sodium, dihydrate, powder” is also a reference to saccharin. Within the context of the oral solution dosage form, reference to “sodium benzoate, powder” is also a reference to benzoic acid and/or sodium benzoate. Within the context of the oral solution dosage form, reference to “methylparaben, powder” is also a reference to methylparaben. Within the context of the oral solution dosage form, reference to “edetate disodium dihydrate” is also a reference to ethylenediaminetetraacetic acid (EDTA).

### Specific Embodiments

The specific embodiments provided below are for illustration purposes only, and do not otherwise limit the scope of the disclosed subject matter, as defined by the claims.

In specific embodiments, the trazodone oral solution is administered for the treatment of major depressive disorder (MIDD).

In specific embodiments, the trazodone oral solution is administered to an adult, at least 18 years old.

In specific embodiments, the trazodone oral solution includes: (i) trazodone; (ii) buffer, (iii) sweetener, (iv) preservative, (v) chelating agent, (vi) colorant, (vii) flavor, and (viii) solvent,

In specific. embodiments, the trazodone oral solution includes: (i) 16.44±1.6 mg/ml trazodone; (ii) 2.40±0.2 mg/ml buffer, (iii) 251.5±25 mg/ml sweetener, (iv) 2±0.2 mg/ml preservative, (v) 3±0.3 mg/ml chelating agent, (vi) 0.01±0.001 mg/ml colorant, (vii) 2±0.2 mg,/ml flavor, and (viii) 739.09±73 mg/ml solvent.

In specific embodiments, the trazodone oral solution includes: (i) 16.44 mg/ml trazodone; (ii) 2.40 mg/ml buffer, (iii) 251.5 mg/ml sweetener, (iv) 2 mg/ml preservative, (v) 3 mg/ml chelating agent, (vi) 0.01 mg/ml colorant, (vii) 2 mg/ml flavor, and (viii) 739.09 mg/ml solvent.

In specific embodiments, the trazodone oral solution includes: (i) trazodone; (ii) citric acid, anhydrous; (iii) sorbitol solution, 70% (w/v); (iv) sodium saccharin; (v) sodium benzoate, powder; (vi) methylparaben, powder; (vii) edetate disodium dihydrate; (viii) FD&C Red No. 40; (ix) cherry flavor; and (x) purified water.

In specific embodiments, the trazodone oral solution includes: (i) 16.44±1.6 mg/ml trazodone; (ii) 2.40±0.2 mg/ml citric acid, anhydrous; (iii) 250±25 mg/ml sorbitol solution, 70% (w/v); (iv) 1.50±0.15 mg/ml sodium saccharin; (v) 1±0.1 mg/ml sodium benzoate, powder; (vi) 1±0.1 mg/ml methylparaben, powder; (vii) 3±0.3 mg/ml edetate disodiurn dihydrate, (viii) 001±0.001 mg/ml FD&C Red No. 40; (ix) 2±0.2 mg;/ml cherry flavor; and (x) 739.0914±75 mg/ml purified water.

In specific embodiments, the trazodone oral solution includes: (i) 16.44 mg/ml trazodone; (ii) 2.40 mg/ml citric acid, anhydrous; (iii) 250 mg/ml sorbitol solution, 70% (w/v); (iv) 1.50 mg/ail sodium saccharin; (v) 1 mg/ml sodium benzoate, powder; (vi) 1 mg/ml methylparaben, powder; (vii) 3 mg/ml edetate disodiuni dihydrate; (viii) 0.01 mg/ml FD&C Red No. 40; (ix) 2 mg/ml cherry flavor; and (x) 739.09 mg/ml purified water.

In specific embodiments, the trazodone oral solution includes trazodone, present as the free base.

In specific embodiments, the trazodone oral solution includes trazodone, present as the hydrochloride salt.

In specific embodiments, the trazodone oral solution includes trazodone, present in 16.44±3 mg/ml based on trazodone hydrochloride.

In specific embodiments, the trazodone oral solution includes trazodone, present in 16.44±1.6 mg/ml based on trazodone hydrochloride.

In specific embodiments, the trazodone oral solution includes trazodone, present in 16.44±1 mg/ml based on trazodone hydrochloride.

In specific embodiments, the trazodone oral solution is manufactured from trazodone, as trazodone hydrochloride.

In specific embodiments, the trazodone oral solution includes citric acid, present in 2.4±0.5 mg/ml.

In specific embodiments, the trazodone oral solution includes citric acid, present in 2.4±0.24 mg/ml.

In specific embodiments, the trazodone oral solution includes citric acid, present in 2.4±0.15 mg/ml.

In specific embodiments, the trazodone oral solution is manufactured from citric acid, as citric acid anhydrous.

In specific embodiments, the trazodone oralsolution includes sorbitol, present in 357±70 mg/ml.

In specific embodiments, the trazodone oral solution includes sorbitol, present in 357±35.7 mg/ml.

In specific embodiments, the trazodone oral solution includes sorbitol, present in 357±17 mg/ml.

In specific embodiments, the trazodone oralsolution is manufactured from sorbitol, as sorbitol solution, 70 percent.

In specific embodiments, the trazodone oral solution includes saccharin, present in 1.5±0.30 mg/ml.

In specific embodiments, the trazodone oral solution includes saccharin, present in 1.5±0,15 mg/ml.

In specific embodiments, thetrazodone oral solution includes saccharin, present in 1.5±0.07 mg./ml.

In specific embodiments, the trazodone oral solution is manufactured from saccharin, as sodium saccharin.

In specific embodiments, the trazodone oral solution includes sodium benzoate, present in 1.0±0.2 mg/ml.

In specific embodiments, the trazodone oral solution includes sodium benzoate, present in 1.0±0.1 mg/ml.

In specific embodiments, the trazodone oral solution includes sodium benzoate, present in 1.0±0.05 mg/ml.

In specific embodiments, the trazodone oral solution is manufactured from sodium benzoate, as sodium benzoate, powder.

In specific embodiments, the trazodone oral solution includes methylparaben, present in 1.0±0.2 mg/ml.

In specific embodiments, the trazodone oralsolution includes methylparaben, present in 1.0 +0.1 mg/ml.

In specific embodiments, the trazodone oral solution includes methylparaben, present in 1.0±0.05 mg/ml.

In specific embodiments, the trazodone oral solution is manufactured from methylparaben, as methylparaben, powder.

In specific embodiments, the trazodone oralsolution includes edetate disodium dihydrate, present in 3.0±0.6 mg/ml.

In specific embodiments, the trazodone oral solution includes edetate disodium dihydrate, present in 3.0±0.3 mg/ml.

In specific embodiments, the trazodone oral solution includes edetate disodium dihydrate, present in 3.0±0.15 mg/ml.

In specific embodiments, the trazodone oral solution is manufactured from EDTA, as edetate disodium dihydrate.

In specific embodiments, the trazodone oral solution has a pH of 3.8−4.7.

In specific embodiments, the trazodone oral solution has a pH of 4.0−4.5.

In specific embodiments, the trazodone oral solution has a pH of 4.1−4.4.

In specific embodiments, the trazodone oral solution has a pH of 4.2−4.3.

In specific embodiments, the trazodone oral solution has a pH of 4.0±0.1.

In specific embodiments, the trazodone oral solution has a pH of 4.1±0.1.

In specific embodiments, the trazodone oral solution has a pH of 4.2±0.25.

In specific embodiments, the trazodone oral solution has a pH of 4.2±0.1.

In specific embodiments, the trazodone oral solution has a pH of 4.3±0.25.

In specific embodiments, the trazodone oral solution has a pH of 4.3±0.1.

In specific embodiments, the trazodone oral solution has a pH of 4.4±0.1.

In specific embodiments, the trazodone oral solution has a pH of 4.5±0.1.

In specific embodiments, the trazodone oralsolution s a specific gravity of 1.055±0.05 g/ml.

In specific embodiments, the trazodone oral solution has a specific gravity of 1.055±0.025

In specific embodiments, the trazodone oral solution has a specific gravity of 1.055±0.01 g/ml.

In specific embodiments, the trazodone oralsolution s a specific gravity of 1.055±0.005 g/ml.

In specific embodiments, the trazodone oral solution has a specific gravity of 1.055 g/ml.

In specific embodiments, the trazodone oral solution has a viscosity of less than 12.5 cP.

In specific embodiments, the trazodone oral solution has a viscosity of less than 10 cP.

In specific embodiments, the trazodone oral solution has a viscosity of less than 9 cP.

In specific embodiments, the trazodone oral solution has a viscosity of less than 8.5 cP.

In specific embodiments, the trazodone oral solution is administered to a human subject to treat major depressive disorder (MDD).

In specific embodiments, the trazodone oral solution is administered to a human subject to treat anxiety disorder.

In specific embodiments, the trazodone oral solution is administered to a human subject to treat difficulty with sleep.

In specific embodiments, the trazodone oral solution is administered such that trazodone, or a pharmaceutically acceptable salt thereof, is orally administered, equivalent to 50±10 mg trazodone free base.

In specific embodiments, the trazodone oral solution is administered such that trazodone, or a pharmaceutically acceptable salt thereof, is orally administered, equivalent to 50±5 mg trazodone free base.

In specific embodiments, the trazodone oral solution is administered such that trazodone, or a pharmaceutically acceptable salt thereof, is orally administered, equivalent to 50±2.5 mg trazodone free base.

In specific embodiments, the trazodone oral solution is administered such that trazodone, or a pharmaceutically acceptable salt thereof, is orally administered, equivalent to 50 mg trazodone free base.

In specific embodiments, the trazodone oral solution is administered such that trazodone, or a pharmaceutically acceptable salt thereof, is orally administered, equivalent to 100±20 mg trazodone free base.

In specific embodiments, the trazodone oral solution is administered such that trazodone, or a pharmaceutically acceptable salt thereof, is orally administered, equivalent to 100±10 mg trazodone free base.

In specific embodiments, the trazodone oral solution is administered such that trazodone, or a pharmaceutically acceptable salt thereof, is orally administered, equivalent to 100±5 mg trazodone free base.

In specific embodiments, the trazodone oral solution is administered such that trazodone, or a pharmaceutically acceptable salt thereof, is orally administered, equivalent to 100 mg trazodone free base.

In specific embodiments, the trazodone oral solution is administered such that trazodone, or a pharmaceutically acceptable salt thereof, is orally administered, equivalent to 150±30 mg trazodone free base.

In specific embodiments, the trazodone oral solution is administered such that trazodone, or a pharmaceutically acceptable salt thereof, is orally administered, equivalent to 150±15 mg trazodone free base.

In specific embodiments, the trazodone oral solution is administered such that trazodone, or a pharmaceutically acceptable salt thereof, is orally administered, equivalent to 150±7.5 mg trazodone free base.

In specific embodiments, the trazodone oral solution is administered such that trazodone, or a pharmaceutically acceptable salt thereof, is orally administered, equivalent to 150 mg trazodone free base.

In specific embodiments, the trazodone oral solution is administered such that trazodone, or a pharmaceutically acceptable salt thereof, is orally administered, equivalent to 300±30 mg trazodone free base.

In specific embodiments, the trazodone oral solution is administered such that trazodone, or a pharmaceutically acceptable salt thereof, is orally administered, equivalent to 300±30 mg trazodone free base.

In specific embodiments, the trazodone oral solution is administered such that trazodone, or a pharmaceutically acceptable salt thereof, is orally administered, equivalent to 300±15 mg trazodone free base.

In specific embodiments, the trazodone oral solution is administered such that trazodone, or a pharmaceutically acceptable salt thereof, is orally administered, equivalent to 300 mg trazodone free base.

In specific embodiments, the trazodone oral solution is administered such that trazodone, or a pharmaceutically acceptable salt thereof, is orally administered, equivalent to 50-300 mg trazodone free base.

In specific embodiments, the trazodone oral solution is administered such that trazodone, or a pharmaceutically acceptable salt thereof, is orally administered, equivalent to 50 mg, 100 mg, 150 mg, or 300 mg trazodone free base.

In specific embodiments, the trazodone oral solution is taken with or without food.

In specific embodiments, the trazodone oral solution is taken with food.

In specific embodiments, the trazodone oral solution is taken without food.

In specific embodiments, the trazodone oral solution is discontinued without being tapered.

In specific embodiments, the trazodone oral solution is packaged in a container.

In specific embodiments, the trazodone oralsolution is packaged in an amber colored polyethylene terephthalate (PET) bottle.

In specific embodiments, the trazodone oral solution is packaged in a glass bottle.

In specific embodiments, the trazodone oral solution is packaged in a high-density polyethylene (HDPE) bottle.

In specific embodiments, the trazodone oral solution is packaged in a low-density polyethylene (HDPE) bottle.

In specific embodiments, the trazodone oral solution is packaged in a polypropylene (PP) bottle.

In specific embodiments, the trazodone oral solution is packaged in a glass or plastic bottle with a child proof closure.

In specific embodiments, the trazodone oral solution is packaged in a glass or plastic bottle and the packaging further includes a syringe or cup, marked in miL, ounces, or both.

In specific embodiments, the trazodone oral solution is packaged in a glass or plastic bottle configured for use to administer multiple doses of topiramate.

In specific embodiments, the trazodone oral solution, while packaged in a container, is free from microbial contamination for a specified period of time (e.g., ≥20 days, ≥30 days, ≥60 days, ≥90 days, ≥180 days, ≥12 months, or ≥24 months) when tested according to <1111>USP-30 NF-25,

In specific embodiments, the trazodone oral solution, while packaged in a container, is free from microbial contamination for an extended period of time encountered with the shipping and storage of the trazodone oral solution.

In specific embodiments, the trazodone oral solution, while packaged in a container, is free from microbial contamination for an extended period of time encountered with the shipping and storage of the trazodone oral solution under ambient conditions, wherein the microbial contamination includes Escherichia coli (E. coli).

In specific embodiments, the trazodone oral solution, while packaged in a container, is free from microbial contamination for an extended period of time encountered with the shipping and storage of the trazodone oral solution under ambient conditions, wherein the microbial contamination includes less than 0.1 wt. % Escherichia coli (E. coli).

In specific embodiments, the trazodone oral solution, while packaged in a container, is free from microbial contamination for an extended period of time encountered with the shipping and storage of the trazodone oral solution under ambient conditions, wherein the microbial contamination includes less than 0.05 wt. % Escherichia coli (E. coli).

In specific embodiments, the trazodone oral solution, while packaged in a container, is free from microbial contamination for an extended period of time encountered with the shipping and storage of the trazodone oral solution under ambient conditions, wherein the microbial contamination includes less than 0.01 wt. % Escherichia coli (E. coli).

In specific embodiments, the trazodone oral solution, while packaged in a container, is free from microbial contamination for an extended period of time encountered with the shipping and storage of the trazodone oral solution under ambient conditions, wherein the microbial contamination includes Burkholderia cepacia complex (BCC).

In specific embodiments, the trazodone oral solution, while packaged in a container, is free from microbial contamination for an extended period of time encountered with the shipping and storage of the trazodone oral solution under ambient conditions, wherein the microbial contamination includes less than 0.1 wt. % Burkholderia cepacia complex (BCC).

In specific embodiments, the trazodone oral solution, while packaged in a container, is free from microbial contamination for an extended period of time encountered with the shipping and storage of the trazodone oral solution under ambient conditions, wherein the microbial contamination includes less than 0.05 wt. % Burkholderia cepacia complex (BCC).

In specific embodiments, the trazodone oral solution, while packaged in a container, is free from microbial contamination for an extended period of time encountered with the shipping and storage of the trazodone oral solution under ambient conditions, wherein the microbial contamination includes less than 0.01 wt. % Burkholderia cepacia complex (BCC).

In specific embodiments, the trazodone oral solution, while packaged in a container, is free from microbial contamination for at least 24 months under ambient conditions.

In specific embodiments, the trazodone oral solution, while packaged in a container, is free from microbial contamination for at least 12 months under ambient conditions.

In specific embodiments, the trazodone oral solution, while packaged in a container, is free from microbial contamination for at least 6 months under ambient conditions.

In specific embodiments, the trazodone oral solution, while packaged in a container, is free from microbial contamination for at least 180 days under ambient conditions,

In specific embodiments, the trazodone oral solution, while packaged in a container, is free from microbial contamination for at least 90 days under ambient conditions.

In specific embodiments, the trazodone oral solution is an immediate release dosage form.

### EXAMPLES

### Example 1

### Components and Composition

| | | | | | Maximum |
|---|---|---|---|---|---|
| Active | | | | Amount/unit | Daily Dose |
| Ingredient | Grade | UNII | Function | (mg/mL) | (MDD) |
| Trazodone | USP | 6E8ZO8LRNM | Active | 16.44 | 657.60 |
| Hydrochloride | | | | | |

| | | | | | Total Daily | | | | ID |
|---|---|---|---|---|---|---|---|---|---|
| Inactive | | | | Amount/ | Intake (TDI) | Maximum Daily | | IID | Dosage |
| Ingredient | | UNII | Function | unit (mg) | based on MDD | Exposure (MDE) | | Route | form |
| Citric Acid | USP | XF417D3PSL | Buffer | 2.40 | 96.00 | 531 | mg | Oral | Solution |
| Anhydrous | | | | | | | | | |
| Sorbitol | USP | 8KW3E207O2 | Sweetener | 250.00 | 10000.00 | 25830 | mg | Oral | Solution |
| Solution, 70 | | | | | | | | | |
| Percent | | | | | | | | | |
| Sodium | USP | SB8ZUX40TY | Sweetener | 1.50 | 60.00 | 2250 | | Oral | Powder; for |
| Saccharin | | | | | | | | | suspension |
| Sodium | USP | OJ245FESEU | Preservative | 1.00 | 40.00 | 120 | mg | Oral | Suspension |
| Benzoate, | | | | | | | | | |
| Powder | | | | | | | | | |
| Methylparaben, | NF | A218C7HI9T | Preservative | 1.00 | 40.00 | 346 | mg | Oral | Solution |
| Powder | | | | | | | | | |
| Edetate | USP | 7FLD91C86K | Chelating | 3.00 | 120.00 | 360 | mg | Oral | Solution |
| Disodium | | | Agent | | | | | | |
| Dihydrate | | | | | | | | | |
| FD and C Red | NA | WZB9127XOA | Colorant | 0.01 | 0.40 | 28 | mg | Oral | Solution |
| No. 40 | | | | | | | | | |
| Cherry Flavor, | NA | NA | Flavor | 2.00 | 80.00 | 290 | MG | Oral | Suspension |
| Nat & Art | | | | | | | | | |

| | | | | | | | | |
|---|---|---|---|---|---|---|---|---|
| Purified Water | USP | NA | Solvent | 739.09 | 29563.60 | NA | NA | NA |

### Example 2

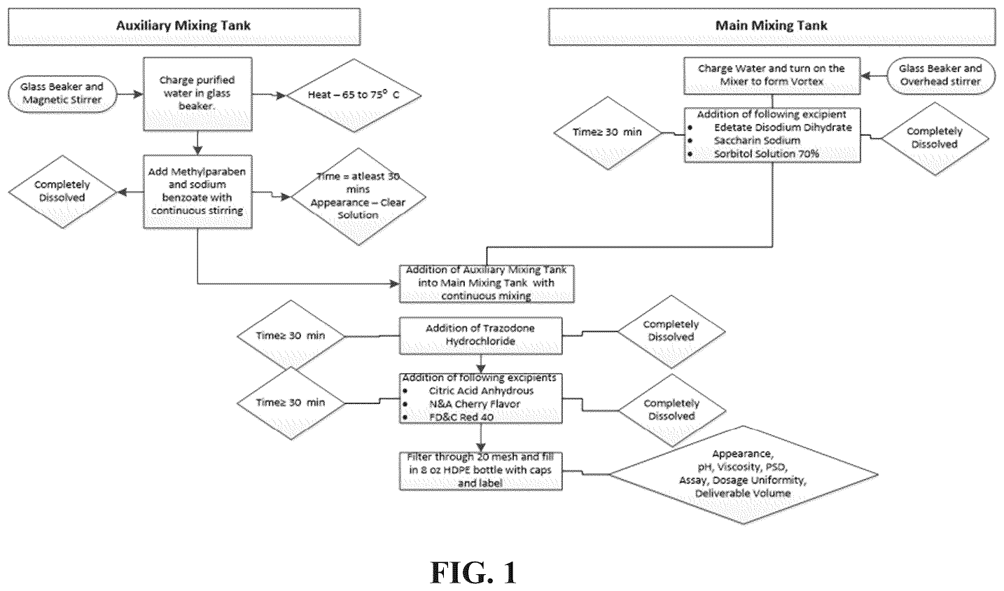

### Manufacturing Process and Controls for a Trazodone Oral Solution as Illustrated in FIG. 1

### Example 3

### Manufacturing Process Outline

1. Main tank mixing
   a. Addition of purified water and mix at 200-300 rpm.
   b. Addition of edetate disodium dihydrate, sodium saccharin, sorbitol solution with continuous mixing until dissolved.
2. Auxiliary tank mixing.
   a. Addition of purified water and mix at 500-700 rpm. Heat at 65°-75° C.
   b. Addition of methylparaben. Mix until dissolved.
   c. Addition of sodium benzoate. Mix until dissolved.
3. Transfer contents of auxiliary tank to main tank. Rinse auxiliary tank with purified water. Continue Mixing at 200-300 rpm and cool to 38°-42° C.
4. Addition of Trazodone HCl with continuous mixing and completely dissolved.
5. Addition of citric acid anhydrous, FD&C Red No 40, Nat & Art Cherry Flavor with continuous mixing and completely dissolved.
6. Q.S the solution with purified water with continuous mixing for 30 minutes.
7. Filter the solution through 20 mesh and filled (236.5 mL solution) in 8 oz HDPE bottle with caps and labeled.

### Physical Properties

Appearance—The product is an orange to red solution free from visible particulate matters with cherry odor.
pH—between 4.0-4.5
Specific gravity—1.055 g/mL.

### Viscosity-<10 cP.

### Example 4

### Pharmacokinetic (PK)

### Pharmacology

### Mechanism of Action

The mechanism of trazodone's antidepressant action is not fully understood, but is thought to be related to its enhancement of serotonergic activity in the CNS, Trazodone is both a selective serotonin reuptake inhibitor (SSRI) and a 5HT2 receptor antagonist and the net result of this action on serotonergic transmission and its role in trazodone's antidepressant effect is unknown.

### Pharmacodynamics

Preclinical studies have shown that trazodone selectively inhibits neuronal reuptake of serotonin (Ki=367 nM) and acts as an antagonist at 5-1-1T-2A (Ki=35.6 nM) serotonin receptors. Trazodone is also an antagonist at several other monoaminergic receptors including 5-HT2B (Ki=78.4 nM), 5-HT2C (Ki=224 nM), α1A. (Ki=153 nM), α2C (Ki =155 nM) receptors and it is a partial agonist at 5- HT1A (Ki=118 nM) receptor.

Trazodone antagonizes alpha 1-adrenergic receptors, a property which may be associated with postural hypotension.

### Pharmacokinetics

### Absorption

In humans, trazodone hydrochloride is absorbed after oral administration without selective localization in any tissue. When trazodone hydrochloride is taken shortly after ingestion of food, there may be an increase in the amount of drug absorbed, a decrease in maximum concentration and a lengthening in the time to maximum concentration. Peak plasma levels occur approximately one hour after dosing when trazodone hydrochloride is taken on an empty stomach or 2 hours after dosing when taken with food.

### Metabolism

In vitro studies in human liver microsomes show that trazodone is metabolized, via oxidative cleavage, to an active metabolite, m-chlorophenvlpipera.zine (mCPP) by CYP3A4. Other metabolic pathways that may be involved in the metabolism of trazodone have not been well characterized. Trazodone is extensively metabolized: less than 1% of an oral dose is excreted unchanged in the urine.

### Elimination

In some patients trazodone may accumulate in the plasma.

### Protein Binding

Trazodone is 89 to 95% protein bound in vitro at concentrations attained with therapeutic doses in humans.

### Example 5

### Stability Testing Report. Room temperature, upright, 180 days.

| | | | | EXP. | BATCH | DATE OF | DATE OF | DATE ON | |
|---|---|---|---|---|---|---|---|---|---|
| PRODUCT NAME: | POTENCY: | CODE: | BATCH # | DATE: | SIZE: | MFG: | PKG: | STABILITY: | COUNT: |
| Trazodone Hydrochloride | 25 mg/mL | 50052 | F0002 (Upri) | N/A | 10.0 LITER | 7 Feb. 2023 | 7 Feb. 2023 | 14 Mar. 2023 | 236 mL per |
| Oral Solution | | | | | | | | | bottle |

| | |
|---|---|
| SITE OF MFG. AND STABILITY STORAGE: | SOURCE OF ACTIVE INGREDIENT: |
| PTS Consulting LLC, 14130 Minnesota | Piramal Pharma Limited, Sy. No. 7-70, 70/1 and 70/2, |
| Avenue, Bonner Springs, KS 66012 | Digwal Village, Kohir Mandal, Sangareddy District, |
| | Telangana, INDIA, Pin code: 502 321 |

| CONTAINER | | LINER | | CLOSURE |
|---|---|---|---|---|
| SIZE: 8 oz | | TYPE: Polyester bonded to aluminum foil wax | | CAP SIZE: 24 mm CRC |
| | | bonded to white lined pulp board (SG 90) | | |
| SHAPE/COLOR: Round/Amber | | MFG: Selig Canada | | RESIN: |
| | | | | CRC Outer Resin: Braskem America PPFPT300F |
| | | | | CRC Inner Resin: Mold-Rite Plastics, LLC CP0001 |
| MATERIAL: PET | | STUFFER: N/A | | MFG.: Mold-Rite Plastics, LLC |
| RESIN: PET Plastic | | MATERIAL: N/A | | COLORANT: Mold-Rite MRPWH01 White Dye |
| COLORANT: Amber | | | | |
| MFG: Alpha Packaging | | MFG: N/A | | ITEM# 42007 |
| ITEM# 40028 | LOT No.: M221568 | ITEM# N/A | LOT No.: N/A | LOT No.: 370922689 |

| | | | | | ACCELERATED (40 ± 2° C./75 ± 5% RH) | | | |
|---|---|---|---|---|---|---|---|---|
| | METHOD | | | INITIAL | 1 | 2 | 3 | 6 |
| TEST | (AM033A) | SPECIFICATION | | T = 0 | MONTH | MONTHS | MONTHS | MONTHS |
| PACKAGE | Visual | Satisfactory (No leaks or distortion of | | Conforms | Conforms | Conforms | Conforms | Conforms |
| INTEGRITY | | container) | | | | | | |
| APPEARANCE | In house | Orange to reddish clear solution free | | Conforms | Conforms | Conforms | Conforms | Conforms |
| | | from visible particulate matter | | | | | | |
| AROMA/TASTE | In house | Cherry aroma (and taste during product | | Conforms | Conforms | Conforms | Conforms | Conforms |
| AND FLAVOR | | development) | | | | | | |
| COLOR (PLATINUM- | ASTM D | Report Pt—Co number. | | Color | Color | Color | Color | Color |
| COBALT SCALE) | 1209-05 | | | Standard | Standard | Standard | Standard | Standard |
| | | | | 200 | 250 | 300 | 350 | 400 |
| WATER LOSS/GAIN | In house | As reported (To evaluate stability trends) | | | −0.1% | −0.4% | −0.3% | |
| IDENTIFICATION A | USP <191> | Yields a yellow-to-orange, curdy | | Conforms | | | | |
| | | precipitate with 0.1N silver nitrate test | | | | | | |
| | | solution. | | | | | | |
| IDENTIFICATION B | | The retention time of the main peak from | | Conforms | | | | |
| | | assay reference standard and the test | | | | | | |
| | | sample solutions correspond. | | | | | | |
| PH | USP <791> | 2.5-3.5 | | 3.2 | 3.3 | 3.2 | 3.2 | 3.2 |
| VISCOSITY | USP <912> | 1.000-1.100 | | 1.0450 | 1.0680 | 1.0680 | 1.0660 | 1.067 |
| SPECIFIC | USP <841> | NMT 10 cP (or mPa*s) | | 3.9 | 4.0 | 4.4 | 4.5 | 3.9 |
| GRAVITY | | | | | | | | |
| ASSAY | USP <621> | 90.0-110.0% of the labeled amount of | | 102.1% | 98.5% | 98.9% | 98.9% | 100.8% |
| | | trazodone hydrochloride | | | | | | |
| | | (C₁₉H₂₂ClN₅O•HCl) | | | | | | |
| IMPURITIES AND | USP <621> | Triazolopyridinone: NMT 0.1% | | ND | ND | ND | ND | ND |
| RELATED | | Trazodone N-oxide: NMT 0.1% | | ND | ND | ND | ND | ND |
| SUBSTANCES 1 | | Deschloro trazodone: NMT 0.1% | | ND | ND | ND | ND | ND |
| | | Trazodone related compound C: NMT 0.1% | | 0.04% | 0.01% | 0.02% | 0.01% | 0.02% |
| | | Trazodone related compound D: NMT 0.1% | | ND | ND | ND | ND | ND |
| | | 4-Ethyl trazodone: NMT 0.1% | | ND | 0.02% | 0.03% | 0.04% | 0.06% |
| | | Trazodone related compound F: NMT 0.25% | | ND | ND | ND | ND | ND |
| | | Trazodone isobutyl ether analog: NMT 0.1% | | ND | ND | ND | ND | ND |
| | | Bispiperazine analog: NMT 0.1% | | ND | ND | ND | ND | ND |
| | | Any individual | RRT = 0.12 | ND | ND | ND | 0.02% | 0.05% |
| | | unspecified | RRT = 0.20 | ND | 0.01% | 0.01% | ND | 0.02% |
| | | degradation | RRT = 0.22 | ND | 0.02% | 0.04% | 0.01% | 0.19% |
| | | product: | RRT = 0.25 | ND | ND | 0.01% | 0.01% | ND |
| | | NMT 1.0% | RRT = 0.28 | ND | ND | 0.02% | 0.01% | ND |
| | | | RRT = 0.29 | ND | ND | ND | 0.01% | BLOQ |
| | | | RRT = 0.30 | ND | ND | 0.06% | 0.10% | ND |
| | | | RRT = 0.55 | 0.04% | 0.12% | 0.20% | 0.24% | 0.33% |
| | | | RRT = 0.84 | 0.03% | 0.07% | 0.09% | 0.10% | 0.12% |
| | | | RRT = 0.93 | ND | ND | 0.01% | 0.01% | ND |
| | | | RRT = 1.29 | ND | 0.01% | 0.01% | 0.02% | 0.02% |
| | | Total degradation products: NMT 2.0% | | 0.10% | 0.26% | 0.50% | 0.53% | 0.73% |
| IMPURITIES AND | USP <621> | Any individual | RRT = 0.15 | ND | ND | ND | 0.17% | 0.62% |
| RELATED | | unspecified | RRT = 0.60 | ND | BLOQ | 0.03% | ND | ND |
| SUBSTANCES 2 | | degradation | RRT = 0.69 | ND | ND | BLOQ | 0.06% | BLOQ |
| | | product: NMT | RRT = 0.73 | ND | ND | ND | ND | 0.64% |
| | | 1.0% | RRT = 0.77 | 0.05% | 0.13% | 0.32% | ND | 0.42% |
| | | | RRT = 0.88 | ND | ND | ND | ND | 0.05% |

| | | | | | | | |
|---|---|---|---|---|---|---|---|
| | | Total degradation products: NMT 2.0% | 0.05% | 0.13% | 0.34% | 0.23% | 1.73% |
| DISSOLUTION | USP <711> | NLT 80% (Q) of the labeled amount of | 97.1% | 96.7% | 96.9% | 96.8% | 97.2% |
| | | trazodone hydrochloride | | | | | |
| | | (C₁₉H₂₂ClN₅O•HCl) is dissolved in 30 | | | | | |
| | | minutes. | | | | | |
| MICROBIAL | USP <61> | Total Aerobic Microbial Count (TAMC): | NG | NG | NG | NG | NG |
| TESTING (USING | | NMT 10² CFU/mL | | | | | |
| MILLIPORE HPC | | Total Combined Yeasts/Molds Count | NG | NG | NG | NG | NG |
| SAMPLERS AND | | (TYMC): | | | | | |
| CONTRACT LAB¹) | | NMT 10¹ CFU/mL | | | | | |
| ESCHERICHIA | USP <62> | Absence of Escherichia coli (in 1 mL of | Absent | | | Absent | Absent |
| COLI TESTING¹ | | solution) | | | | | |
| BURKHOLDERIA | USP <60> | Absence of any BCC organisms | Absent | | | Absent | Absent |
| CEPACIA | | | | | | | |
| COMPLEX (BCC) | | | | | | | |
| TESTING¹ | | | | | | | |
| ANTIMICROBIAL | USP <51> | Bacteria: NLT 1.0 log reduction from the | Conforms | | | Conforms | Conforms |
| EFFECTIVENESS | | initial count at 14 days and no increase | | | | | |
| TEST¹ | | from 14 days' count at 28 day | | | | | |
| | | Yeast and Mold: No increase from the | Conforms | | | Conforms | Conforms |
| | | initial calculated count at 14 and 28 days | | | | | |
| SODIUM | USP <341> | Sodium Benzoate: 80-120% | 103.9% | 100.8% | 101.3% | 100.6% | 97.8% |
| BENZOATE | | | | | | | |
| (PRESERVATIVE) | | | | | | | |

| | | | | | |
|---|---|---|---|---|---|
| NO. OF UNITS TO BE SAMPLED | 2 | 1 | 1 | 2 | 2 |
| ACTUAL UNITS SAMPLED | 2 | 1 | 1 | 2 | 2 |
| SCHEDULE DATE FOR REMOVAL (OR INITIAL PLACEMENT) | | 13 Apr. | 13 May | 12 Jun. | 11 Sep. |
| | | 2023 | 2023 | 2023 | 2023 |
| ACTUAL DATE OF REMOVAL (OR INITIAL PLACEMENT) | | 13 Apr. | 15 May | 12 Jun. | 11 Sep. |
| | | 2023 | 2023 | 2023 | 2023 |
| TEST COMPLETION DATE | 19 Apr. | 26 Apr. | 31 May | 8 Aug. | 2 Nov. |
| | 2023 | 2023 | 2023 | 2023 | 2023 |

### Example 6

### Stability Testing Report. 180 days.

| | | | | EXP. | BATCH | DATE OF | DATE OF | DATE ON | |
|---|---|---|---|---|---|---|---|---|---|
| PRODUCT NAME: | POTENCY: | CODE: | BATCH # | DATE: | SIZE: | MFG: | PKG: | STABILITY: | COUNT: |
| Trazodone | 25 mg/mL | 50052 | F0002 (Upri) | N/A | 10.0 LITER | 7 Feb. 2023 | | 14 Mar. 2023 | 236 mL per |
| Hydrochloride Oral | | | | | | | | | bottle |

| | |
|---|---|
| SITE OF MFG. AND STABILITY STORAGE: | SOURCE OF ACTIVE INGREDIENT: |
| PTS Consulting LLC, 14130 Minnesota | Piramal Pharma Limited, Sy. No. 7-70, 70/1 and 70/2, |
| Avenue, Bonner Springs, KS 66012 | Digwal Village, Kohir Mandal, Sangareddy District, |
| | Telangana, INDIA, Pin code: 502 321 |

| CONTAINER | | LINER | | CLOSURE |
|---|---|---|---|---|
| SIZE: 8 oz | | TYPE: Polyester bonded to aluminum foil wax | | CAP SIZE: 24 mm CRC |
| | | bonded to white lined pulp board (SG 90) | | |
| SHAPE/COLOR: Round/Amber | | MFG: Selig Canada | | RESIN: |
| | | | | CRC Outer Resin: Braskem America PPFPT300F |
| | | | | CRC Inner Resin: Mold-Rite Plastics, LLC CP0001 |
| MATERIAL: PET | | STUFFER: N/A | | MFG.: Mold-Rite Plastics, LLC |
| RESIN: PET Plastic | | MATERIAL: N/A | | COLORANT: Mold-Rite MRPWH01 White Dye |
| COLORANT: Amber | | | | |
| MFG: Alpha Packaging | | MFG: N/A | | ITEM# 42007 |
| ITEM# 40028 | LOT No.: M221568 | ITEM# N/A | LOT No.: N/A | LOT No.: 370922689 |

| | | | | | ACCELERATED (40 ± 2° C./75 ± 5% RH) | | | |
|---|---|---|---|---|---|---|---|---|
| | METHOD | | | INITIAL | 1 | 2 | 3 | 6 |
| TEST | (AM033A) | SPECIFICATION | | T = 0 | MONTH | MONTHS | MONTHS | MONTHS |
| PACKAGE | Visual | Satisfactory (No leaks or distortion of | | Conforms | Conforms | Conforms | Conforms | Conforms |
| INTEGRITY | | container) | | | | | | |
| APPEARANCE | In house | Orange to reddish clear solution free | | Conforms | Conforms | Conforms | Conforms | Conforms |
| | | from visible particulate matter | | | | | | |
| AROMA/TASTE | In house | Cherry aroma (and taste during product | | Conforms | Conforms | Conforms | Conforms | Conforms |
| AND FLAVOR | | development) | | | | | | |
| COLOR | ASTM D | Report Pt-Co number. | | Color | Color | Color | Color | Color |
| (PLATINUM- | 1209-05 | | | Standard | Standard | Standard | Standard | Standard |
| COBALT SCALE) | | | | 200 | 250 | 300 | 350 | 400 |
| WATER LOSS/GAIN | In house | As reported (To evaluate stability | | | −0.1% | −0.4% | −0.3% | |
| | | trends) | | | | | | |
| IDENTIFICATION A | USP <191> | Yields a yellow-to-orange, curdy | | Conforms | | | | |
| | | precipitate with 0.1N silver nitrate test | | | | | | |
| | | solution. | | | | | | |
| IDENTIFICATION B | | The retention time of the main peak | | Conforms | | | | |
| | | from assay reference standard and the | | | | | | |
| | | test sample solutions correspond. | | | | | | |
| PH | USP <791> | 2.5-3.5 | | 3.2 | 3.3 | 3.2 | 3.2 | 3.2 |
| VISCOSITY | USP <912> | 1.000-1.100 | | 1.0450 | 1.0680 | 1.0680 | 1.0660 | 1.067 |
| SPECIFIC | USP <841> | NMT 10 cP (or mPa*s) | | 3.9 | 4.0 | 4.4 | 4.5 | 3.9 |
| ASSAY | USP <621> | 90.0-110.0% of the labeled amount of | | 102.1% | 98.5% | 98.9% | 98.9% | 100.8% |
| | | trazodone hydrochloride | | | | | | |
| | | (C₁₉H₂₂ClN₅O•HCl) | | | | | | |
| IMPURITIES AND | USP <621> | Triazolopyridinone: NMT 0.1% | | ND | ND | ND | ND | ND |
| RELATED | | Trazodone N-oxide: NMT 0.1% | | ND | ND | ND | ND | ND |
| SUBSTANCES 1 | | Deschloro trazodone: NMT 0.1% | | ND | ND | ND | ND | ND |
| | | Trazodone related compound C: NMT 0.1% | | 0.04% | 0.01% | 0.02% | 0.01% | 0.02% |
| | | Trazodone related compound D: NMT 0.1% | | ND | ND | ND | ND | ND |
| | | 4-Ethyl trazodone: NMT 0.1% | | ND | 0.02% | 0.03% | 0.04% | 0.06% |
| | | Trazodone related compound F: NMT 0.25% | | ND | ND | ND | ND | ND |
| | | Trazodone isobutyl ether analog: NMT 0.1% | | ND | ND | ND | ND | ND |
| | | Bispiperazine analog: NMT 0.1% | | ND | ND | ND | ND | ND |
| | | Any | RRT = 0.12 | ND | ND | ND | 0.02% | 0.05% |
| | | individual | RRT = 0.20 | ND | 0.01% | 0.01% | ND | 0.02% |
| | | unspecified | RRT = 0.22 | ND | 0.02% | 0.04% | 0.01% | 0.19% |
| | | degradation | RRT = 0.25 | ND | ND | 0.01% | 0.01% | ND |
| | | product: NMT | RRT = 0.28 | ND | ND | 0.02% | 0.01% | ND |
| | | 1.0% | RRT = 0.29 | ND | ND | ND | 0.01% | BLOQ |
| | | | RRT = 0.30 | ND | ND | 0.06% | 0.10% | ND |
| | | | RRT = 0.55 | 0.04% | 0.12% | 0.20% | 0.24% | 0.33% |
| | | | RRT = 0.84 | 0.03% | 0.07% | 0.09% | 0.10% | 0.12% |
| | | | RRT = 0.93 | ND | ND | 0.01% | 0.01% | ND |
| | | | RRT = 1.29 | ND | 0.01% | 0.01% | 0.02% | 0.02% |
| | | Total degradation products: NMT 2.0% | | 0.10% | 0.26% | 0.50% | 0.53% | 0.73% |
| IMPURITIES AND | USP <621> | Any | RRT = 0.15 | ND | ND | ND | 0.17% | 0.62% |
| RELATED | | individual | RRT = 0.60 | ND | BLOQ | 0.03% | ND | ND |
| SUBSTANCES 2 | | unspecified | RRT = 0.69 | ND | ND | BLOQ | 0.06% | BLOQ |
| | | degradation | RRT = 0.73 | ND | ND | ND | ND | 0.64% |
| | | product: NMT | RRT = 0.77 | 0.05% | 0.13% | 0.32% | ND | 0.42% |
| | | 1.0% | RRT = 0.88 | ND | ND | ND | ND | 0.05% |

| | | | | | | | |
|---|---|---|---|---|---|---|---|
| | | Total degradation products: NMT 2.0% | 0.05% | 0.13% | 0.34% | 0.23% | 1.73% |
| DISSOLUTION | USP <711> | NLT 80% (Q) of the labeled amount of | 97.1% | 96.7% | 96.9% | 96.8% | 97.2% |
| | | trazodone hydrochloride | | | | | |
| | | (C₁₉H₂₂ClN₅O•HCl) is dissolved in 30 | | | | | |
| | | minutes. | | | | | |
| MICROBIAL | USP <61> | Total Aerobic Microbial Count | NG | NG | NG | NG | NG |
| TESTING (USING | | (TAMC): | | | | | |
| MILLIPORE HPC | | NMT 10² CFU/mL | | | | | |
| SAMPLERS AND | | Total Combined Yeasts/Molds Count | NG | NG | NG | NG | NG |
| CONTRACT LAB¹) | | (TYMC): | | | | | |
| | | NMT 10¹ CFU/mL | | | | | |
| ESCHERICHIA | USP <62> | Absence of Escherichia coli (in 1 mL | Absent | | | Absent | Absent |
| COLI TESTING¹ | | of solution) | | | | | |
| BURKHOLDERIA | USP <60> | Absence of any BCC organisms | Absent | | | Absent | Absent |
| CEPACIA | | | | | | | |
| COMPLEX (BCC) | | | | | | | |
| TESTING¹ | | | | | | | |
| ANTIMICROBIAL | USP <51> | Bacteria: NLT 1.0 log reduction from | Conforms | | | Conforms | Conforms |
| EFFECTIVENESS | | the initial count at 14 days and no | | | | | |
| TEST¹ | | increase from 14 days' count at 28 day | | | | | |
| | | Yeast and Mold: No increase from the | Conforms | | | Conforms | Conforms |
| | | initial calculated count at 14 and 28 | | | | | |
| | | days | | | | | |
| SODIUM | USP <341> | Sodium Benzoate: 80-120% | 103.9% | 100.8% | 101.3% | 100.6% | 97.8% |
| BENZOATE | | | | | | | |
| (PRESERVATIVE) | | | | | | | |

| | | | | | |
|---|---|---|---|---|---|
| NO. OF UNITS TO BE SAMPLED | 2 | 1 | 1 | 2 | 2 |
| ACTUAL UNITS SAMPLED | 2 | 1 | 1 | 2 | 2 |
| SCHEDULE DATE FOR REMOVAL (OR INITIAL PLACEMENT) | | 13 Apr. | 13 May | 12 Jun. | 11 Sep. |
| | | 2023 | 2023 | 2023 | 2023 |
| ACTUAL DATE OF REMOVAL (OR INITIAL PLACEMENT) | | 13 Apr. | 15 May | 12 Jun. | 11 Sep. |
| | | 2023 | 2023 | 2023 | 2023 |
| TEST COMPLETION DATE | 19 Apr. | 26 Apr. | 31 May | 8 Aug. | 2 Nov. |
| | 2023 | 2023 | 2023 | 2023 | 2023 |

What is claimed:

1. An oral solution comprising:
(i) trazodone hydrochloride;
(ii) citric acid;
(iii) sorbitol;
(iv) sodium saccharin;
(v) sodium benzoate;
(vi) methylparaben;
(vii) edetate disodium dihydrate;
(viii) FD & C Red No. 40;
(ix) cherry flavor; and
(x) water.

2. The oral solution of claim 1, wherein the trazodone hydrochloride is present in 16.44±1.6 mg/ml.

3. The oral solution of claim 1, wherein the trazodone hydrochloride is present in 16.44 mg/ml.

4. The oral solution of claim 1, wherein the citric acid is present in 2.4±0.24 mg/ml.

5. The oral solution of claim 1, wherein the citric acid is present in 2.4 mg/ml.

6. The oral solution of claim 1, wherein the sorbitol is present in 357±35.7 mg/ml.

7. The oral solution of claim 1, wherein the sorbitol is present in 357 mg/ml.

8. The oral solution of claim 1, wherein the sodium saccharin is present in 1.5±0.15 mg/ml.

9. The oral solution of claim 1, wherein the sodium saccharin is present in 1.5 mg/ml.

10. The oral solution of claim 1, wherein the sodium benzoate is present in 1.0±0.1 mg/ml.

11. The oral solution of claim 1, wherein the sodium benzoate is present in 1.0 mg/ml.

12. The oral solution of claim 1, wherein the methylparaben is present in 1.0±0.1 mg/ml.

13. The oral solution of claim 1, wherein the methylparaben is present in 1.0 mg/ml.

14. The oral solution of claim 1, wherein the edetate disodium dihydrate is present in 3.0±0.3 mg/ml.

15. The oral solution of claim 1, wherein the edetate disodium dihydrate is present in 3.0 mg/ml.

16. The oral solution of claim 1, which is substantially free from visible particulate matters.

17. The oral solution of claim 1, having a pH of 4.0-4.5.

18. The oral solution of claim 1, having a specific gravity of 1.055 g/ml.

19. The oral solution of claim 1, having a viscosity of less than 10 cP.

20. A method of treating in a human subject major depressive disorder (MDD), the method comprising administering to the human subject in need thereof an affective amount of the oral solution of claim 1.
